(19) United States
(12) Patent Application Publication
(10) Pub. No.: US 2009/0210001 A1
(43) Pub. Date: Aug. 20, 2009

(54) DEVICE FOR IMPLANTING AN OCCLUSION DEVICE IN THE HEART

(75) Inventor: Peter Osypka, Rheinfelden-Herten (DE)

Correspondence Address: VOLPE AND KOENIG, P.C., UNITED PLAZA, SUITE 1600, 30 SOUTH 17TH STREET, PHILADELPHIA, PA, 19103, US

(73) Assignee: Peter Osypka Stiftung Stiftung des burgerlichen Rechts, Grenzach-Wyhlen (DE)

(21) Appl. No.: 12/372,020

(22) Filed: Feb. 17, 2009

(30) Foreign Application Priority Data
Feb. 16, 2008 (DE) 102008009525.7-43

(51) Int. Cl. A61B 17/12

(52) U.S. Cl. 606/213

(57) ABSTRACT

A device (1) used for the implantation of an occlusion (2) in an undesired opening in a septum (3) in a heart (4) is provided and has a catheter (5) with a displacement element (7) (pusher) that can move in a longitudinal direction of the catheter in the interior and on whose distal end the occlusion (2) is fixed detachably and can be brought into its position of use, whereupon the displacement element (7) is detached again from the occlusion (2). The occlusion (2) connected to the displacement element (7) is arranged for the implantation process in the distal end region of the catheter (5) and is completely or at least partially enclosed by this end region.

### CROSS-REFERENCE TO RELATED APPLICATIONS

This application claims the benefit of German Application DE 2008 009 525.7, filed Feb. 16, 2008, which is incorporated herein by reference as if fully set forth.

### BACKGROUND

The invention relates to a device for implanting an occlusion device in an undesired opening of the septum between the two atria of a heart with a catheter and with a displacement element that can move in this catheter in the direction of longitudinal extent of the catheter, and the occlusion can be mounted detachably or fixed detachably at the distal end of this displacement element and can be brought from the distal end of the catheter into its position of use by the displacement movement and then can be separated from the displacement element.

A comparable device is known from U.S. Pat. No. 6,334,864 B1. Therein it is described that the occlusion formed, as a rule, from a metal mesh, is to be shifted across the entire length of the catheter into the heart, which, due to the length of such a catheter or catheter tube and the curves to be traversed is also therefore associated with problems, because during the displacement movement that moves the occlusion through the catheter, the metal mesh of this occlusion rubs against the inside of the catheter that is often made from plastic or is coated with plastic and therefore there is the risk that plastic particles are rubbed off and brought into the heart.

### SUMMARY

Thus, there is the objective of creating a device of the type noted above that reduces or rules out the risk of rubbed-off particles from the interior of the catheter.

To meet this objective, the device according to the invention has the occlusion connected to the displacement element (pusher) which is arranged before and during the implantation process in the interior of the catheter in the distal end region of the catheter and is enclosed by this end region completely or at least partially.

Through this arrangement, for the introduction of the occlusion out of the catheter at the delivery location, only a relatively small displacement movement is necessary with a correspondingly low risk of rubbed-off particles. It is sufficient to introduce the catheter together with the occlusion located in its distal end region into the heart and then to shift the occlusion from the distal end of the catheter into the opening in the septum in the interior of the heart and therefore to bring it into the position of use, without having to shift the occlusion over the entire length of the catheter.

The catheter can contain a flexible coil or can be formed by such a coil. This produces the advantage of a large degree of flexibility for the catheter that can here contain the displacement element located in an inner lumen of the catheter and that can nevertheless be well adapted to different and changing directions of a blood vessel.

A very especially advantageous configuration of the invention can provide that the distal end of the catheter is formed by a holder tube that is arranged there and that is connected to the rest of the catheter—continuing in the direction of longitudinal extent of the catheter—and holds the occlusion at least partially in itself, such that, in the starting position, the distal end of the occlusion matches the distal end of the holder tube or is set back relative to the end of the holder tube.

Through such a holder tube supplementing or extending the catheter and belonging to the catheter, an especially good adaptation to an occlusion element is possible that obtains its final shape only after being pushed out from the catheter, that is, at first it is introduced and held within the catheter in an elongated shape. During the implantation, the holder tube allows the catheter to be able to easily follow a blood vessel together with the occlusion that is located in it and that is deformed relative to its position of use, that is, allows it to perform corresponding bending movements.

Here it is especially favorable if the holder tube holds the occlusion and the attachment position of this occlusion to the displacement element. In this way, the entire occlusion and its coupling position or position of the detachable mounting is housed on the displacement element in the holder tube that can be easily adapted with its tube structure to the outside of the occlusion element and its attachment position.

It is preferred when the holder tube has a cross section that exceeds the cross section of the rest of the catheter. The catheter can have correspondingly narrow and thin dimensions, while the holder tube can have a somewhat larger cross-sectional extent for holding the occlusion.

The introduction of the catheter and the occlusion transported by it into the interior of the heart can be made easier when the device has a guide wire or a guide wire belongs to the device, wherein this guide wire can be introduced before the catheter, for example, into a blood vessel, and a guide for this guide wire is provided on the catheter and/or on the holder tube at least close to or at the distal end. With the help of a guide wire, the catheter can be introduced smoothly and easily into the heart, without, however, requiring relative movements between the catheter and the occlusion, because the occlusion is located at the distal end of the catheter or the holder tube.

The guide for the guide wire can be an opening that runs approximately in the radial direction or at an angle and that is oriented perpendicular to or passes through the wall of the holder tube. In this way, it is also possible that the guide for the guide wire is formed by a sleeve or eyelet having a corresponding opening. If the holder tube itself has a corresponding perforation as an opening for the guide wire, the attachment of a corresponding counter guide is unnecessary. An eyelet or sleeve, however, improves the guiding.

In this way, one preferred embodiment can provide that an opening used as a guide through the wall of the holder tube is arranged at a distance to its end in the region of the distal end of the occlusion held before or during the implantation of the holder tube and the part of the guide wire running between this opening and the distal end of the tube runs in the interior of this tube along the distance of the distal end of the occlusion element to the distal end of the holder tube. Thus it is possible, in the starting position, to arrange the distal end of the occlusion at a distance to the distal end of the catheter or the holder tube and to arrange the guide wire in this region of the catheter or holder tube in the interior of this holder tube that therefore remains free from the occlusion. Thus, the guide wire can pass outward from the catheter or holder tube at a position that is essentially free from the occlusion, so that the displacement of the catheter and the holder tube does not lead to additional friction between the guide wire and occlusion along the guide wire.

It may be preferable when a sleeve-like guide runs on the outside of at least the holder tube and the guide wire continues after emerging from the guide sleeve on the outside or via an inlet opening in the interior of the catheter. In this way, friction between the occlusion and guide wire during the implantation process can also be avoided.

The displacement element can be shifted with its attachment position for the occlusion past the distal end of the catheter and/or the holder tube, then the occlusion can be connected to the displacement element, in particular, can be connected by a threaded connection, and then can be drawn into the implantation position in the interior of the distal end of the catheter or its holder tube.

Because a detachable or separable connection is provided between the displacement element and occlusion, this connection can also be produced initially in that the attachment position of the displacement element is fed through a correspondingly wide displacement past the distal end of the catheter, where the occlusion can be easily attached. Through retraction, the occlusion then reaches its position of use provided during the implantation process. This can be performed by a doctor during preparation in a run-up to the use of the device or directly before the use of the device. In the latter case, the occlusion could also be delivered separately from the catheter and brought into a corresponding position of use only immediately before use.

The holder tube holding the occlusion before and during the implantation can be sealed fluid-tight on the inside relative to the adjacent catheter and the displacement element can be shifted fluid-tight relative to this seal. Thus, it is largely prevented that blood penetrates into the catheter during the implantation.

In addition, it is possible that a filling made from a blood-compatible fluid, in particular, a saline solution, is filled in the end of the catheter used for holding the occlusion or in the holder tube. Therefore, the introduction of air during the implantation process can be prevented with greater reliability.

The holder tube can have small holes through which, when the fluid or saline solution pushing out the air is filled before the implantation, the air can be forced out from this holder tube, wherein the holes are selected to be so small that the air can be forced through them but the fluid remains in the interior of the holder tube due to its surface tension or the size of its molecules. Such an arrangement requires the fluid or saline solution to be filled with pressure so that the air is actually also forced through the relatively small holes. The reliability of forcing out the air completely is correspondingly great.

Another configuration of the device can provide that the holder tube has a pre-set curvature and can be steered through rotation of the catheter about is longitudinal axis. The catheter with the holder tube can be easily pushed accordingly toward the interior of the heart, wherein, due to the pre-set curvature of the holder tube, a guide wire can be eliminated, but the implantation can also be supported with the help of a guide wire.

Another configuration of the device, in particular, a device having a guide wire can provide that the guide wire has, at its distal end, an extension or a stop that has an enlarged cross section relative to the guide opening or a guide sleeve or eyelet and that the holder tube can be bent by a tensile force on the guide wire through its eccentric arrangement relative to the center of the holder tube. Thus, the guide wire can obtain an additional function, because it can be used for steering the catheter during the implantation, for example, when the catheter and the holder tube has already been pushed across a large distance of the guide wire and then a deflection of the distal end appears advantageous.

The holder tube can be made from plastic or as a thin-walled metal sleeve or as a coil or as a mesh, optionally with a coating. This holder tube belonging to the catheter is stable on one side and flexible on the other side accordingly.

In this way, the edge of the distal end of the holder tube can be rounded or beveled for simplifying the introduction or the entire end of the holder tube can be beveled and/or can have a soft, flexible form—similar to a hollow needle. This also promotes the introduction of the catheter and its holder tube with the occlusion located therein.

Primarily for the combination of individual or several of the features and measures described above, a device is produced for implanting an occlusion in an undesired opening in the interior of the heart at which, for the relative movement between the occlusion and the catheter or holder tube holding it, only a very short displacement movement is required, that is, the risk of rubbed-off particles from the interior of the catheter through the displacement of the occlusion into its position of use is essentially prevented.

### BRIEF DESCRIPTION OF THE DRAWINGS

Below, embodiments of the invention are described in greater detail with reference to the drawing. Shown in partially schematic representation are:

FIG. 1 is a view of a device according to the invention for implanting an occlusion in an undesired opening in the septum between the two atria of a heart, wherein a catheter belonging to the device is introduced through a blood vessel into the heart and the occlusion is pushed out into its position of use with the help of a displacement element arranged in the interior of the catheter but is not yet separated from the displacement element,

FIG. 2 is a side view of the device according to the invention, wherein the displacement element with the attachment position for the occlusion is pushed past the distal end of the catheter and a holder tube located there before the occlusion is fixed at this position,

FIG. 3 is, at an enlarged scale, a longitudinal section view of the distal end of the catheter and the holder tube with the pushed displacement element according to FIG. 2,

FIG. 4 is a view according to FIG. 2 after the attachment and screwing of the occlusion on the distal end of the displacement element and a thread provided there,

FIG. 5 is a view according to FIG. 4 after the displacement element and the occlusion have been partially retracted and a part of the occlusion still projects past the distal end of the catheter or the holder tube,

FIG. 6 is a view according to FIGS. 2, 4, and 5 in which the occlusion and the displacement element are arranged in the position of use, that is, in the position at which the implantation can be performed,

FIG. 7 is a view according to FIG. 3, that is, a longitudinal section view of the distal end of the catheter and the holder tube with the occlusion element arranged therein and deformed by the holder tube, wherein this occlusion element can be pushed from this position according to FIG. 1 in the direction away from a handle of the device into the opening of the heart, in order to allow, for example, with two interconnected screens, contact at the edges of the opening in the interior of the heart on both sides of the septum,

FIG. 8 is a view of a modified embodiment from that shown in FIG. 6, wherein a guide wire is provided that runs essentially along the outside of the catheter and interacts with a guide opening close to the distal end of the holder tube,

FIG. 9 is, at an enlarged scale, a longitudinal section view of the distal end of the catheter and the holder tube with the deformed occlusion located therein and the arrangement of the guide wire that enters into the interior of the holder tube in the region of the distal end of the retracted occlusion through the wall of the holder tube,

FIG. 10 is a view, corresponding to FIG. 9, of a modified embodiment in which the guide wire runs through a sleeve-like guide on the outside of the holder tube,

FIG. 11 is a side view of the distal end of the catheter and the holder tube with a modified arrangement of the guide wire that transitions at the junction between the holder tube and the catheter into the interior of the catheter,

FIG. 12 is a side view of the distal end of the catheter and the holder tube analogous to FIG. 10, wherein, on the distal end of the guide wire there is a stop that can interact with the distal end of the sleeve-shaped guide, and

FIG. 13 is a view corresponding to FIG. 12 after retracting the guide wire until the stop interacts with the distal end of the sleeve-like guide such that a tensile force on the guide wire leads to a curving of the distal end of the catheter or the holder tube.

### DETAILED DESCRIPTION OF THE PREFERRED EMBODIMENTS

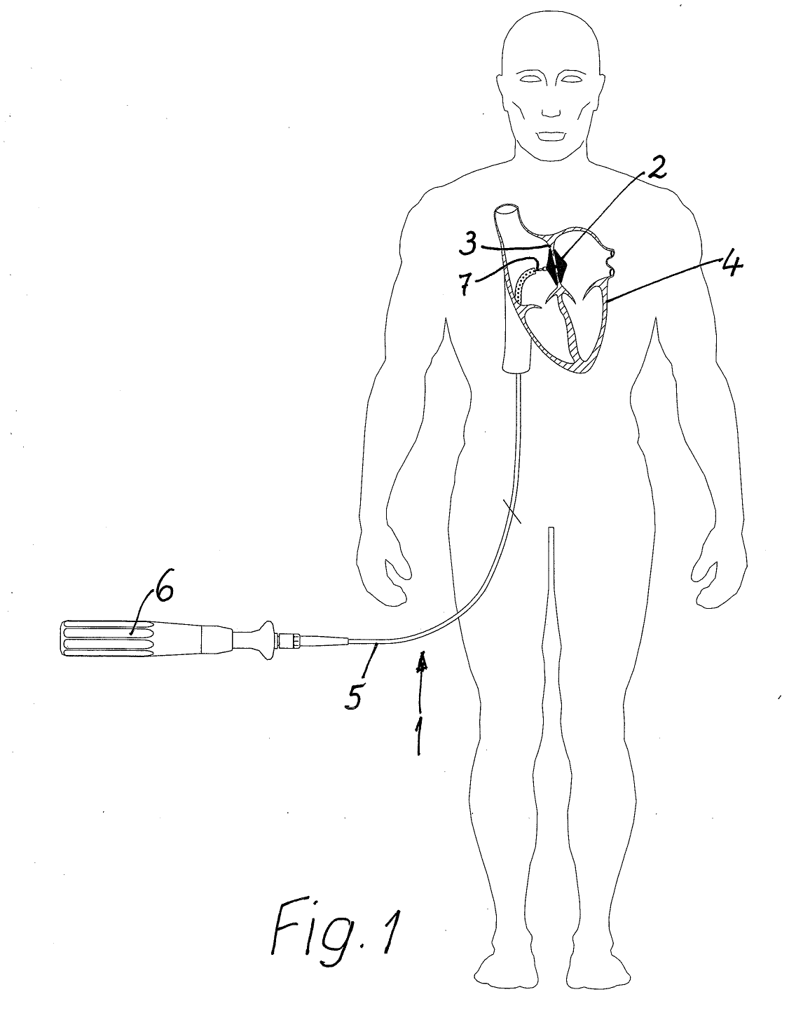

A device designated as a whole with 1 is used for implanting an occlusion 2 in an undesired opening of the septum 3 between the two atria of a heart 4. Such occlusions 2 are known and have, according to FIGS. 1 and 4 in the position of use, the shape of two somewhat spaced apart, oppositely arranged screens that hold the cardiac septum 3 and the undesired opening between themselves.

An essential component of the device 1 is a catheter designated as a whole with 5 that has a handle 6 for its activation according to FIGS. 1, 2, 4 to 6, and 8.

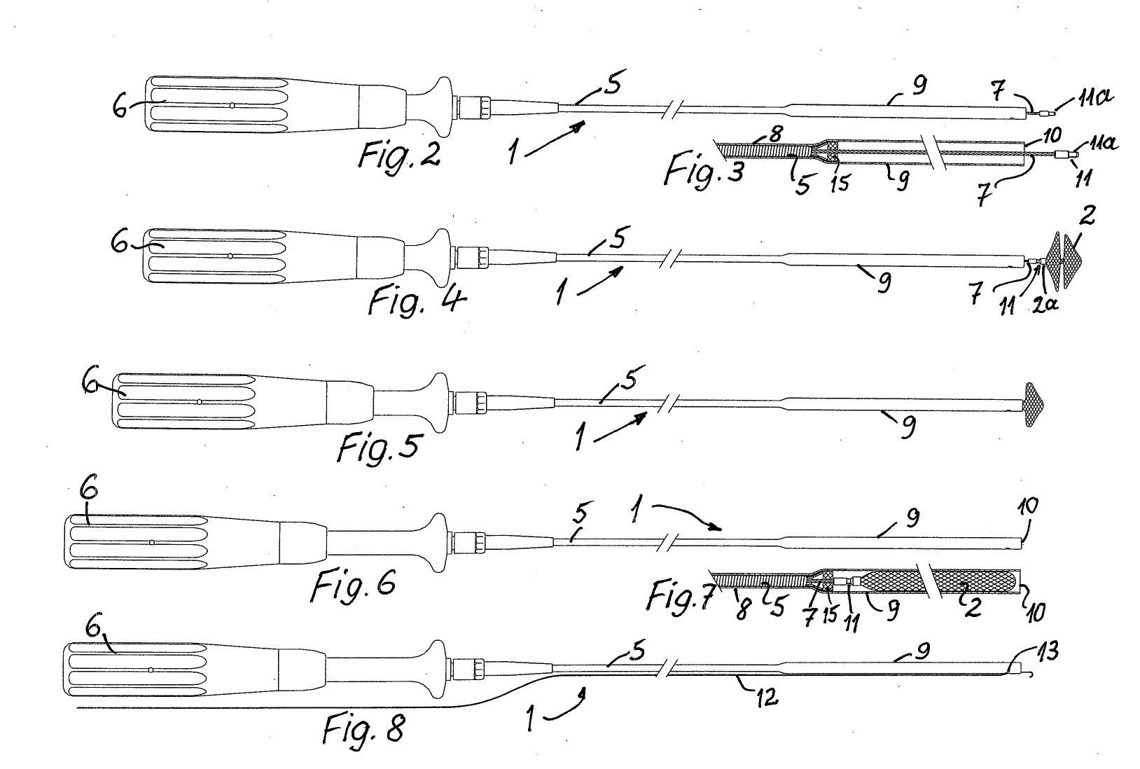

Furthermore, a displacement element 7 is included with the device 1. This displacement element can move in the interior of the catheter 5 in the direction of the longitudinal extent of the catheter and can be seen primarily in FIGS. 3 and 4 and is formed as a flexible wire in the embodiment, that is, it is flexible, and can follow the bending of the catheter 5 during the implantation. At the distal end of the displacement element 7, according to FIGS. 2 to 4 and also 9 and 10, the occlusion 2 can be connected detachably or fixed detachably and can be brought from the starting position provided in FIGS. 6 to 13 into the position of use shown in FIG. 1 by the mentioned displacement movement. If this position according to FIG. 1 is reached, the displacement element 7 can be separated or detached and retracted again from the occlusion 2.

So that a large displacement path is not needed for the displacement of the occlusion 2 and there is no risk that the mesh-shaped, metallic occlusion 2 will remove or rub off particles from the inside of the catheter 5 during such displacement, the occlusion 2 connected to the displacement element 7 is arranged in the interior of the catheter at the front of and, for the implantation process according to FIGS. 6 to 11, in the distal end region of the catheter 5. The displacement movement required for inserting the occlusion 2 into the opening of the septum 3 in the heart 4 during the implantation process is accordingly short.

In FIGS. 3, 7, 9, and 10, it is indicated that the catheter 5 contains a flexible coil or is formed by such a coil that can still be coated with a tube 8, so that the catheter 5 has the desired high degree of flexibility, in order to be able to follow changes in direction in the vessels through which the implantation is performed.

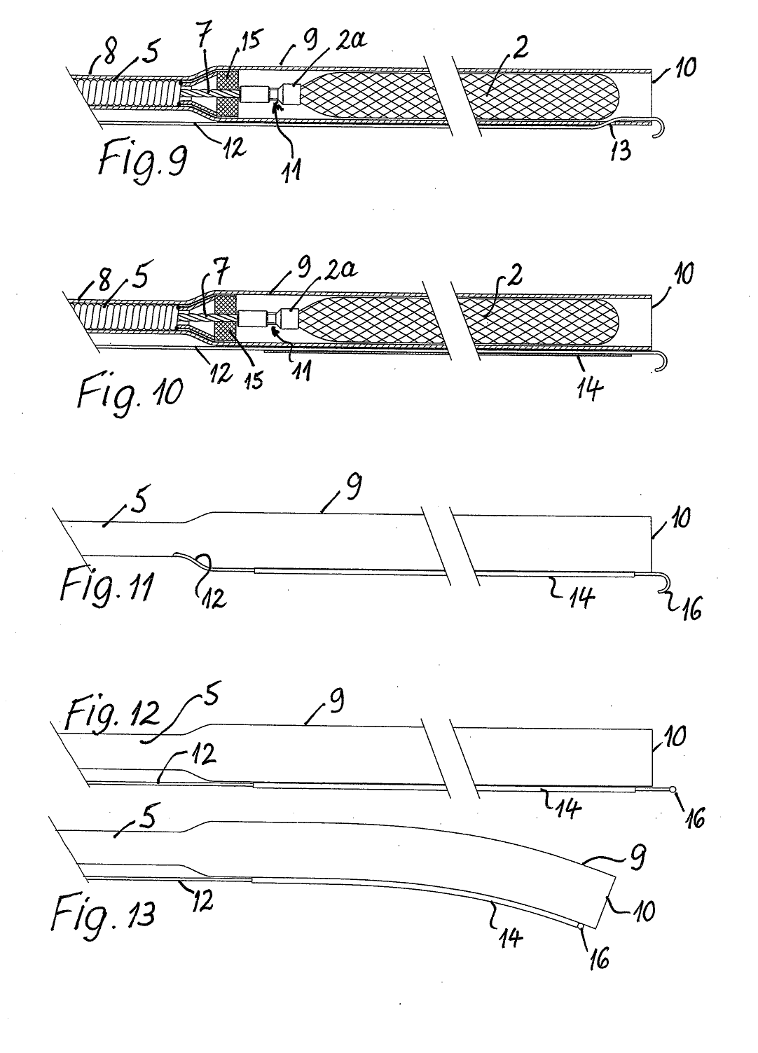

The distal end of the catheter 5 is formed in all of the embodiments by a holder tube 9 that is arranged there and that can be the continuation of the tube 8 according to FIGS. 8, 9, and 10. In each case, this holder tube 9 forming the distal end of the catheter 5 is connected to the remainder of the catheter 5 and holds the occlusion 2 in the starting position such that the distal end of the occlusion 2 in this starting position agrees with the distal end 10 of the holder tube 9 or preferably is set back relative to this end 10 according to FIGS. 9 and 10. In FIGS. 9 and 10, one sees a slight distance between the distal end of the occlusion 2 located in the holder tube 9 from the distal end 10 of the holder tube 9. In this way it is guaranteed that the occlusion 2 remains well protected in the interior of the holder tube 9 during the implantation of the catheter 5 and thus also the occlusion 2 and is surrounded over its entire length.

In this way, the holder tube 9 holds the occlusion 2 and its attachment position 11 to the displacement element 7, that is, the position 11 at which the occlusion 2 is connected and fixed detachably to the displacement element 7 is also located in the interior of the holder tube 9.

In this way, with reference to the drawing and, in particular, FIGS. 9 and 10, it also becomes clear that the holder tube 9 has a cross section that exceeds the cross section of the remainder of the catheter 5, so that it surrounds and can hold a correspondingly voluminous occlusion 2 well. The attachment position 11 can also be seen in FIG. 3, wherein the occlusion 2 has not yet been connected and, for example, a pin-shaped part 11a can be provided there with an external thread that fits in an internal thread on a sleeve 2a that belongs to the occlusion 2 and holds its mesh together.

In the embodiments according to FIGS. 8 to 13, it is shown that the device 1 can have a guide wire 12, that is, a guide wire 12 is provided with the device 1, wherein this guide wire typically can be introduced before the catheter 5 into a corresponding blood vessel and into the heart 4. A guide for this guide wire 12 is provided on the catheter 5 or preferably on the holder tube 9 at least close to or at the distal end 10. This guide can have a different form and allows the catheter 5 to follow the previously implanted guide wire into the heart 4 for its implantation.

In the embodiment according to FIGS. 8 and 9, the guide for the guide wire 12 is an opening 13 that runs approximately in the radial direction or at an angle and that is oriented perpendicular to and passes through the wall of the holder tube 9. This is clear primarily in FIG. 9 by a double change in direction of the guide wire 12 that initially runs on the outside of the catheter 5 and the holder tube 9 and then transitions through the opening 13 into the interior of the holder tube 9, runs along the inside, and then grips over the end 10.

In the embodiments according to FIGS. 10 to 13, the guide for the guide wire 12 is formed by a flexible sleeve 14 or eyelet having a corresponding opening. This produces a long, mutual guide, but also requires this sleeve 14.

According to FIG. 9, the opening 13 used as a guide through the wall of the holder tube 9 is arranged at a distance to its distal end 10 in the region of the distal end of the occlusion 2 held and enclosed by the holder tube 9 before or during the implantation and the part of the guide wire 12 running between this opening 13 and the distal end 10 of the holder tube 9 runs in the region of this distance of the distal end of the occlusion element 2 to the distal end 10 in the interior of the holder tube 9. Therefore, friction between the occlusion 2 and the guide wire 12 during the implantation of the catheter 5 can be prevented.

In FIGS. 10 and 12 it is shown that the sleeve-like guide or guide sleeve 14 runs on the outside of the holder tube 9 and the guide wire 12 continues on the outside along the catheter 5 after emerging from this guide sleeve 14—seen from the distal end of the holder tube 9.

FIG. 11 shows a comparable arrangement in which, however, the guide wire 12 is transferred after emerging from the guide sleeve 14 through an inlet opening into the interior of the catheter 5 and continues there up to the proximal end.

In FIGS. 2 to 7 it is shown that the displacement element 7 is shifted or can be shifted with its attachment position 11 past the distal end 10 of the catheter 5 or the holder tube 9, so that the occlusion 2 can be connected easily to the displacement element 7 and the attachment position 11, for example, as already mentioned, can be connected via a threaded connection. FIG. 4 shows this arrangement. According to FIGS. 5 and 6, the occlusion 2 then can be performed in the implantation position in the interior of the distal end of the catheter 5 or the holder tube 9 through a corresponding retraction movement on the displacement element 7 or an advance movement of the catheter 5 relative to the displacement element 7, in order to produce the starting position according to FIGS. 6 and 7 for the implantation process. An analogous situation applies for the embodiments according to FIGS. 8 to 13.

In this way, this connection of the occlusion 2 with the occlusion element 7 can already be realized at the completion of the device 1 or else shortly before the implantation process.

In FIGS. 3, 7, 9, and 10, it is shown that the holder tube 9 holding the occlusion 2 before and during the implantation is sealed fluid-tight on the inside relative to the catheter 5 connecting to the proximal end and the displacement element or the pusher 7 can be displaced fluid-tight relative to the seal 15 used for this purpose. In the mentioned figures, one clearly sees how the displacement element 7 formed as a flexible wire passes through the seal 15 that here contacts the displacement element 7 in a sealing manner.

Therefore it is possible that a filling made from a blood-compatible fluid, for example, made from a saline solution, is filled into the end of the catheter 5 used for holding the occlusion 2, in the embodiments into the holder tube 9, in order to force air out. Here, the holder tube 9 can have small holes which are too small to be identified in the drawing. Through these holes, the air can be forced out from the holder tube 9 when the fluid or saline solution forcing the air out is filled before the implantation, because the filling is performed under pressure, wherein the holes are selected so small that air can be forced out through them, but the fluid remains in the interior of the holder tube 9 due to its surface tension and/or the molecule sizes.

In FIG. 13, a curvature of the holder tube 9 is shown that could be a pre-set curvature relative to the diagram of FIG. 13, in order to allow steering through rotation of the catheter 5 about its longitudinal axis during the implantation process. Such a pre-set curvature would allow this steering even if no guide wire 12 were present.

In FIGS. 12 and 13 it is shown that the guide wire 12 can have, on its distal end, an extension or a stop 16 that has an enlarged cross section in comparison with the guide opening 13 or the guide sleeve 14 or a guide sleeve, that is, does not pass through this opening 13 or sleeve 14 or eyelet. Therefore, the holder tube 9 can be curved or bent through a tensile force applied to the guide wire 12 through its eccentric arrangement relative to the center of the holder tube 9 in the way that can be seen in FIG. 13, in order to allow steering during the implantation.

The holder tube 9 shown in the embodiments is preferably made from plastic, but can also be made from a thin-walled, correspondingly shaped and connected metal sheet, that is, formed as a thin-walled metal sleeve. Furthermore, the holder tube 9 can also be formed as a coil or as a mesh optionally with a coating, in order to have a correspondingly good strength. The edge of the distal end 10 of the holder tube 9 can also be rounded or beveled in the different embodiments, in order to simplify the insertion at narrow points or branches of the blood vessels.

In addition, it is possible that the entire end 10 is not arranged, as shown in the figures, perpendicular to the longitudinal center axis, but instead at an angle to this axis, that is, angled similar to a hollow needle. In this way, this end 10 and its edge can preferably have a soft, flexible form that also simplifies the implantation process.

The device 1 is used for the implantation of an occlusion 2 in an undesired opening in a septum 3 in a heart 4 and has a catheter 5 with a displacement element 7 (pusher) that can move in the longitudinal direction of the catheter in the interior and on whose distal end the occlusion 2 is fixed detachably and can be brought into its position of use, where the displacement element 7 is detached again from the occlusion 2. Here, it is provided that the occlusion 2 connected to the displacement element 7 is arranged for the implantation process in the distal end region of the catheter 5 and is completely or at least partially enclosed by this end region.

1. Device (1) for implanting an occlusion (2) in an undesired opening of the septum (3) between the two atria of a heart (4), comprising a catheter (5) with a displacement element (7) that can move in the catheter in a direction of longitudinal extent of the catheter and at whose distal end the occlusion (2) is placed detachably or fixed detachably and can be brought into its position of use by a displacement movement from the distal end of the catheter (5) and then can be separated from the displacement element (7), the occlusion (2) connected to the displacement element (7) is arranged in an interior of the catheter in a distal end region of the catheter (5) before and during the implantation process and is completely or at least partially enclosed by the distal end region.

2. Device according to claim 1, wherein the catheter (5) comprises a flexible coil.

3. Device according to claim 1, wherein the distal end of the catheter (5) is formed by a holder tube (9) that is arranged there and that is connected to a remainder of the catheter (5) and holds the occlusion (2) at least partially, such that the distal end of the occlusion (2) in a starting position is even with or set back relative to the distal end (10) of the holder tube (9).

4. Device according to claim 3, wherein the holder tube (9) holds the occlusion (2) and an attachment position (11) of the occlusion to the displacement element (7).

5. Device according to claim 3, wherein the holder tube (9) has a cross section that exceeds a cross section of the remainder of the catheter (5).

6. Device according to claim 3, further comprising a guide wire (12) that can be introduced, for example, into a blood vessel before the catheter (5), a guide for the guide wire (12) is provided on the catheter (5) or on the holder tube (9) proximate to the distal end (10).

7. Device according to claim 6, wherein the guide is an opening (13) that extends approximately in a radial direction or at an angle and that is oriented perpendicular to or passes through the wall of the holder tube (9).

8. Device according to claim 6, wherein the guide for the guide wire (12) is formed by a sleeve (14) or eyelet having a corresponding opening.

9. Device according to claim 6, wherein an opening (13) used as a guide through the wall of the holder tube (9) is arranged at a distance from the end (10) in a region of a distal end of the occlusion (2) held by the holder tube (9) before or during the implantation and the part of the guide wire (12) running between the opening (13) and the distal end (10) of the holder tube (9) runs in an interior of the holder tube along a distance of the distal end of the occlusion element (2) to the distal end (10) of the holder tube.

10. Device according to claim 6, wherein the guide is a sleeve-like guide (14) and runs on an outside of at least the holder tube (9) and the guide wire (12) continues after emerging from the guide sleeve (14) on the outside or via an inlet opening in an interior of the catheter (5).

11. Device according to claim 1, wherein the displacement element (7) can be pushed with an occlusion attachment position (11) thereof past the distal end (10) of the catheter (5), then the occlusion (2) can be connected to the displacement element (7), in particular, connected via a threaded connection, and then drawn into an implantation position in an interior of the distal end of the catheter (5).

12. Device according to claim 1, wherein the catheter includes a holder tube (9) at the distal end thereof, the holder tube (9) holds the occlusion (2) before and during the implantation and is closed fluid-tight on an inside relative to an adjacent portion of the catheter (5) and the displacement element (7) can be shifted fluid-tight relative to a seal (15).

13. Device according to claim 12, wherein a filling made from a blood-compatible fluid is filled into an end of the catheter (5) used for holding the occlusion (2).

14. Device according to claim 13, wherein the holder tube (9) has small holes through which, when filling the fluid, allows air to be forced out before the implantation, the holes are sized such that the air can be forced out through them but the fluid remains in an interior of the holder tube (9) due to its surface tension.

15. Device according to claim 1, wherein the catheter includes a holder tube (9) at the distal end thereof, the holder tube (9) has a pre-set curvature and can be steered by rotating the catheter (5) about a longitudinal axis thereof.

16. Device according to claim 6, wherein the guide wire (12) has, on a distal end thereof, an extension or a stop (16) that has an enlarged cross section in comparison with the guide, which comprises an opening (13) or a guide sleeve (14) or eyelet on the holder tube, and the holder tube (9) can be bent by a tensile force on the guide wire (12) through its eccentric arrangement relative to a middle of the holder tube (9).

17. Device according to claim 3, wherein the holder tube (9) is made from plastic, a thin-walled metal sleeve, a coil or a mesh.

18. Device according to claim 3, wherein at least an edge of the distal end (10) of the holder tube (9) is at least one of rounded or beveled or has a soft, flexible form.

19. Device according to claim 3, wherein an entirety of the distal end (10) of the holder tube (9) is at least one of rounded or beveled or has a soft, flexible form.
